SEQUENCE LISTING (7 sequences)

SEQ ID NO: 1 - protein, 15 aa, Artificial
  Peptide derived from protein tau
KKVAVVRTPPKSPSS

SEQ ID NO: 2 - protein, 15 aa, Artificial
  Peptide derived from protein APP
VEVDAAVTPEERHLS

SEQ ID NO: 3 - protein, 15 aa, Artificial
  Peptide derived from protein cdc25 thr48p
VCPDVPRTPVGKFLG

SEQ ID NO: 4 - protein, 15 aa, Artificial
  Peptide derived from protein tau
ATRIPAKTPPAPKTP

SEQ ID NO: 5 - protein, 15 aa, Artificial
  Peptide derived from protein tau
SGYSSPGSPGTPGSR

SEQ ID NO: 6 - protein, 15 aa, Artificial
  Peptide derived from protein tau
GSRSRTPSLPTPPTR

SEQ ID NO: 7 - protein, 15 aa, Artificial
  Peptide derived from protein tau
DTSPRHLSNVSSTGS

(19) United States
(12) Patent Application Publication
(10) Pub. No.: US 2005/0221391 A1
(43) Pub. Date: Oct. 6, 2005

(54) Reagents and methods for identification of binding agents

(76) Inventor: Peter Davies, Rye, NY (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/332,666

(22) PCT Filed: Jul. 11, 2001
(86) PCT No.: PCT/US01/21859

§ 371 Date: Oct. 14, 2003

Related U.S. Application Data
(60) Provisional application No. 60/217,604, filed on Jul. 11, 2000.

(51) Int. Cl. G01N 33/53; G01N 33/567

(52) U.S. Cl. 435/7.2

(57) ABSTRACT

A method for identifying a desired binding agent that interferes with the interaction between a protein, protein fragment, polypeptide or a peptide and a binding surrogate. The method comprises combining the protein, protein fragment, polypeptide or peptide, the binding surrogate and a binding agent. Detecting a decrease in the interaction between the protein, protein fragment, polypeptide or peptide from the binding surrogate indicates that the binding agent interferes with the interaction. Proteins useful in the method include the tau protein phosphorylated at threonine (231), the Amyloid Precursor Protein (APP) phosphorylated at threonine (668 and cdc25 phosphorylated at threonine (48). Compounds identified by the method are useful in the treatment of Alzheimers' disease and cancer.

### FIELD OF THE INVENTION

This invention relates to reagents and methods for discovery of compounds that bind to specific sites on tau, the Amyloid Precursor Protein (APP), and cdc25. Such compounds are useful in the treatment of Alzheimer's disease and cancer, for example.

### DESCRIPTION OF THE RELATED ART

Tau is the major component of the paired helical filaments (PHF) that make up the neurofibrillary tangles characteristic of the brains of patients with Alzheimer's Disease (“AD”). The processes by which normal tau protein is modified to form PHP are not completely understood, but there is general agreement that these processes involve both abnormal phosphorylation of tau, and changes in the conformation of the protein. The Amyloid Precursor Protein (APP) is the precursor of the 40 to 42 amino acid peptide that is deposited in the neuritic plaques of Alzheimer's disease. A large body of recent work suggests that changes in the proteolytic cleavage of the APP occur in Alzheimer's disease such that excessive amounts of the 40-42 amino acid peptide are produced. The nature of these changes in processing are poorly understood.

Recent work has suggested that the existence of a common feature between tau and APP, which may explain why both proteins become abnormal and contribute to the development of the pathology of Alzheimer's disease. Both tau and APP have an unusual structural feature, a reverse beta turn, in regions that are known to be phosphorylated. This structure, especially when phosphorylated, serves as a recognition site for the binding of a number of proteins that regulate tau and APP function, and aberrant binding of proteins to these sites probably contributes to the development of abnormal conformations of tau, and to alterations in proteolysis of APP.

Neurofibrillary tangles in Alzheimer's disease contain tan phosphorylated at threonine 231, that can be demonstrated by the staining of brain tissues from such cases using any one of several different monoclonal antibodies (Vincent, et al. Mitotic mechanisms in Alzheimer's Disease? Journal of Cell Biology, 132, 413-425, 1996; Vincent, et al. Mitotic phosphoepitopes precede paired helical filaments in Alzheimer's Disease. Neurobiol Aging. 19, 287-296, 1998). Phosphorylation at this site has also been directly demonstrated by the sequencing of tau isolated from purified PBF (Hasegawa et al. Protein sequence and mass spectrometric analyses of tau in the Alzheimer's disease brain J. Biol. Chem. 267, 17047-17054, 1992). In 1998, Jicha et al showed that the conformation of tau in the region of threonine 231 was also abnormal, using a monoclonal antibody called TG3, the binding of which is sensitive both to phosphorylation of tau and to the conformation of the protein (Jicha, et al. Conformation and phosphorylation dependent antibody recognizing the paired helical filaments of Alzheimer's Disease. J. Neurochem. 69, 2087-2095, 1997). A number of different studies have shown that this region of the tau molecule is the site for binding of several proteins, including fyn (Lee, et al. Tau interacts with src-family non-receptor tyrosine kinases. J Cell Sci. 111, 3167-3177, 1998), PLC-gamma (Hwang, et al. Activation of phospholipase C-gamma by the concerted action of tau proteins and arachidonic acid. J Biol Chem. 271, 18342-18349, 1996), PPI (Liao, et al. Protein phosphatase 1 is targeted to microtubules by the microtubule-associated protein Tau. J Biol Chem. 273, 21901-21908, 1998), PP2A (Sontag et al. Mumby M C. Regulation of the phosphorylation state and microtubule-binding activity of Tau by protein phosphatase 2A. Neuron. 17, 1201-1207, 1996), kinesin (Jancsik et al. Tau proteins bind to kinesin and modulate its activation by microtubules. Neurobiology . 4, 417-429, 1996; Johnson et al. Tau protein in normal and Alzheimer's disease brain: an update. Alzheimer's Disease Review, 3, 125-141, 1998) and Pin1 (Lu, et al. The prolyl isomerase Pin1 restores the biological function of Alzheimer-associated phosphorylated tau. Nature, 399, 784-788, 1999). For one of these proteins, Pin1, there is direct evidence that binding is dependent on phosphorylation of threonine 231, and binding of the other proteins is likely to be similarly affected. Binding of Pin1 to phosphorylated tau has also been demonstrated to alter the conformation of tau, and it is probable that this may also occur on binding of other proteins. While low levels of phosphorylation of tau at this site probably do occur in the normal brain, the extent of phosphorylation of threonine 231 is greatly increased in Alzheimer's disease. Prevention of the deleterious consequences of this phosphorylation is one aim of the methods described in this application.

That structural similarities exist between the region of tau around threonine 231 (“thr231”) and that surrounding threonine 668 of APP (“thr668”) was first demonstrated by the binding of a monoclonal antibody, MC2, to both sites following phosphorylation of the threonine. Similarly, the protein Pin1 binds to phosphopeptides derived from both proteins, respectively. This data suggests significant structural similarity between these two regions from proteins which otherwise have little or no sequence homology. It is likely that these regions adopt similar conformations, and there is published evidence that the region of the APP protein around thr668 adopts a reverse beta turn structure (Shen, et al. The essential mitotic peptidyl-prolyl isomerase Pin1 binds and regulates mitosis-specific phosphoproteins. Genes Dev. 12, 706-720. 1998). Evidence has also been obtained that the same structural feature is found near thr231 of tau.

It is known that conformational changes in tau occur prior to PHP formation, and not as a result of filament formation. Those skilled in the art generally agree that specific conformational changes of tau are among the earliest detectable changes within neurons of the AD brain (Hyman, et al. Alz-50 antibody recognizes Alzheimer-related neuronal changes. Ann Neurol 23; 371-379, 1988; Carmel, et al. The structural basis of monoclonal antibody Alz50's selectivity for Alzheimer's disease pathology. J. Biol Chem. 271, 32789-32795, 1996; Jicha, et al.: Alz-50 and MC-1, a new monoclonal antibody raised to paired helical filaments, recognize conformational epitopes on recombinant tau. J. Neuroscience Research, 48, 128-132, 1997; Jicha, et al. Sequence requirements for formation of conformational variants of tau similar to those found in Alzheimer's Disease. J Neurosci. Res, 55, 713-723, 1999; Jicha, et al. Conformation and phosphorylation dependent antibody recognizing the paired helical filaments of Alzheimer's Disease. J. Neurochem. 69, 2087-2095, 1997). It has also been demonstrated that phosphorylation is related to these conformation changes.

One specific phosphorylation of tau, on thr231, also appears to occur very early in the process of AD, probably at or close to the time at which conformational changes are detectable. Specific monoclonal antibodies detecting phosphothreonine 231 of tau have been utilized to demonstrate conformational changes in the phosphorylated tau, suggesting a link between this phosphorylation and conformation changes in the protein (Jicha, et al. A Conformation and phosphorylation dependent antibody recognizing the paired helical filaments of Alzheimer's Disease. 3. Neurochem. 69, 2087-2095, 1997). In addition, phosphorylation at thr231 has been directly demonstrated by sequencing tau isolated from purified PHBF (Hasegawa, et al. Protein sequence and mass spectrometric analyses of tau in the Alzheimer's disease brain. J. Biol. Chem. 267, 17047-17054, 1992).

A number of different studies have shown that this region of the tau molecule is the site for binding of several proteins, including fyn (Lee, et al. Tau interacts with src-family non-receptor tyrosine kinases. J Cell Sci. 111, 3167-3177, 1998), phospholipase C (“PLC”)-gamma (Hwang, et al. Activation of phospholipase C-gamma by the concerted action of tau proteins and arachidonic acid. J Biol Chem. 271, 18342-18349, 1996), protein phosphatase I (“PPI”) (Liao et al. Gundersen G G. Protein phosphatase 1 is targeted to microtubules by the microtubule-associated protein Tau. J Biol Chem. 273, 21901-21908, 1998), protein phosphatase 2A (“PP2A”; Sontag, et al. Regulation of the phosphorylation state and mnicrotubule-binding activity of Tau by protein phosphatase 2A. Neuron 17, 1201-1207, 1996), kinesin (Jancsik et al. Tau proteins bind to kinesin and modulate its activation by microtubules. Neurobiology 4, 417-429, 1996; Johnson, et al. Tau protein in normal and Alzheimer's disease brain: an update. Alzheimer's Disease Review, 3, 125-141, 1998) and Pin1 (Lu, et al. The prolyl isomerase Pin1 restores the biological function of Alzheimer-associated phosphorylated tau. Nature, 399, 784-788, 1999).

Thr668 is found in the C-terminal region of APP, a region of the protein believed to be intracellular, with the bulk of the protein protruding through the cell membrane into the extracellular space. The intracellular C-terminal region has been reported to interact with several proteins, including Go and Fe65 (Kroenke et al. Solution conformations of a peptide containing the cytoplasmic domain sequence of the beta amyloid precursor protein. Biochemistry 36, 8145-8152, 1997). Phosphorylation of this region has also been reported to alter proteolytic processing of APP and secretion of the 40-42 amino acid peptide deposited in the plaques of Alzheimer's disease (Kroenke, supra; Weaver, et al. Conformational Requirements For The Monoclonal Antibody TG3 Reactivity And Specificity For Alzheimer's Disease Elucidated Through NMR Spectroscopy. Society for Neuroscience, Abstract #448.10, 1999; Russo, et al. Fe65 and the protein network centered around the cytosolic domain of the Alzheimer's beta-amyloid precursor protein. FEBS Letters. 434, 1-7, 1998).

The “Amyloid Cascade Hypothesis”, dominant in this field for several years, has proposed that deposition of beta-amyloid in the brain led to neuronal degeneration and tangle formation, direct experimental evidence for this has been difficult to obtain (Selkoe D J. Cell biology of the beta-amyloid precursor protein and the genetics of Alzheimer's disease. Cold Spring Harbor Symposia on Quantitative Biology. 61:587-596, 1996; Davies, P.: Neuronal abnormalities, not amyloid, are the cause of dementia in Alzheimer's Disease. pages 327-333 In ALZHEIMER's DISEASE. Edited by Katzman, et al. Raven Press, New York, 1994). Most notably, transgenic mice which develop huge numbers of beta-amyloid deposits in the brain at a young age, fail to develop evidence of significant tau pathology, and no evidence of neurofibrillary tangle formation, even when aged (Holcomb, et al. Accelerated Alzheimer-type phenotype in transgenic mice carrying both mutant amyloid precursor protein and presenilin 1 transgenes. Nature Medicine 4:97-100, 1998). As a result, the amyloid cascade hypothesis has recently been modified. It has been suggested that intracellular accumulation of beta-amyloid (rather than extracellular deposition in plaques leads to AD pathology, (Skovronsky, et al. Detection of a novel intraneuronal pool of insoluble amyloid beta protein that accumulates with time in culture. J Cell Biol 141:1031-1039, 1998; Yang, et al. Intracellular accumulation of insoluble, newly synthesized Abeta42 in amyloid precursor protein-transfected cells that have been treated with Abeta 1-42. J Biol Chem 274:20650-20656, 1999).

Beta-amyloid is formed by the cleavage of a larger precursor, the amyloid precursor protein or APP. This protein is abundant in neurons, and much of what is synthesized within neurons is cleaved near the center of the beta-amyloid peptide region, with secretion of the larger N-terminal fragment of the molecule, and presumably intracellular retention of the smaller C-terminal fragment (Selkoe D J. Cell biology of the beta-amyloid precursor protein and the genetics of Alzheimer's disease. Cold Spring Harbor Symposia on Quantitative Biology. 61:587-596, 1996;. Selkoe D J. Translating cell biology into therapeutic advances in Alzheimer's disease. Nature 399:A23-31, 1999). This cleavage is catalysed by a currently unidentified protease called alpha-secretase. As alpha-secretase cleavage occurs within the beta-amyloid domain, production and deposition of this peptide is impossible following this secretory processing. Two different cleavages of the APP are required to liberate the beta-amyloid peptide, a beta-secretase cleavage to generate the N-terminus of the beta-amyloid peptide, and one or more gamma secretase cleavages to generate the C-terminus. At least some of the beta-amyloid peptide generated in normal cells is secreted. The fate of the remainder of the APP molecule is unclear. Much attention has been focused on the beta and gamma secretases in recent years, and the beta secretase was recently cloned (Vassar, et al. Beta-secretase cleavage of Alzheimer's amyloid precursor protein by the transmembrane aspartic protease BACE. Science 286:735-741, 1999). Mechanisms that might control the production of the beta-amyloid from APP have attracted a great deal of interest in recent years, and a novel potential control mechanism was recently identified.

The C-terminus of APP is known to be phosphorylated in brain of animals, and in cell culture (Oishi, et al. The cytoplasmic domain of Alzheimer's amyloid precursor protein is phosphorylated at Thr654, Ser655, and Thr668 in adult rat brain and cultured cells. Mol Med 3:111-123, 1997; Suzuld, et al. Cell cycle-dependent regulation of the phosphorylation and metabolism of the Alzheimer amyloid precursor protein. EMBO J. 13:1114-1122, 1994). It has been suggested that phosphorylation of the C-terminus of APP controls the rate of cleavage, at least at the alpha-secretase site (Caporaso, et al. Protein phosphorylation regulates secretion of Alzheimer/A4 amyloid precursor protein. PNAS 89:3055-3059, 1992). There is little available information in this area, mainly due to the fact that specific monoclonal antibodies to phosphorylation sites in APP were not available until recently.

Thr668 has been reported to be a preferred site for cdc2 phosphorylation (Suzuki, et al. Cell cycle-dependent regulation of the phosphorylation and metabolism of the Alzheimer amyloid precursor protein. EMBO J. 13:1114-1122, 1994). Interestingly, cdc2 (also called cdk1) is the most efficient of all kinases studied at phosphorylating thr231 of tau (Jicha, et al. A Conformation and phosphorylation dependent antibody recognizing the paired helical filaments of Alzheimer's Disease. J. Neurochem. 69, 2087-2095, 1997). There have been suggestions that cdc2 activity is up regulated in the brains of AD patients, and that cdc2 may be associated with neurofibrillary tangles, especially in the early stages of formation (Vincent, et al. Aberrant expression of mitotic cdc2/cyclin b1 kinase in degenerating neurons of Alzheimers disease brain. J Neurosci. 17:3588-3598, 1997). This kinase is better known as a critical regulator of the cell cycle, and the appearance of cdc2 in post-mitotic cells such as neurons was unexpected. This work has led to the so-called “Mitotic Hypothesis” of Alzheimer's disease, which proposes that aberrant activation of the cell cycle in post-mitotic neurons is responsible for neurofibrillary tangle formation and cellular degeneration (McShea, et al. Abnormal expression of the cell cycle regulators p16 and cdk4 in Alzheimers-disease. Am J Path. 150:1933-1939, 1997; Nagy, et al. Cell cycle markers in the hippocampus in Alzheimers-disease. Acta Neuropath. 94:6-15, 1997; Nagy, et al. Expression of cell division markers in the hippocampus in Alzheimer's disease and other neurodegenerative conditions. Acta Neuropath. 93:294-300, 1997; Illenberger, et al. The endogenous and cell-cycle dependent phosphorylation of tau protein in living cell: implications for Alzhiemer's disease. Molec Bio Cell 9, 1495-1512, 1998).

Recent work has suggested that a second key regulator of the cell cycle may also participate in tangle formation and neurodegeneration. Pin1 is a prolyl isomerase which binds to and isomerizes serine/threonine-proline bonds in proteins, only if the serine or threonine is phosphorylated. It is essential for normal mitosis in all eukaryotic cells (Yaffe, et al. Sequence-specific and phosphorylation-dependent proline isomerization: a potential mitotic regulatory mechanism Science 278:1957-1960, 1997; Lu, et al. A human peptidyl-prolyl isomerase essential for regulation of mitosis. Nature 380:544-547, 1996). Sequences recognized by Pin1 in peptide substrates were strikingly similar to sequences in tau, and it was discovered that Pin1 binds strongly to the tau sequence surrounding thr231 but only when the threonine is phosphorylated (Lu, et al. The prolyl isomerase Pin1 restores the biological function of Alzheimer-associated phosphorylated tau. Nature, 399, 784-788, 1999).

Pin1 is responsible for regulating protein kinase activation timing during mitosis to ensure orderly progression through the cell cycle. A major target of Pin1 activity in cells undergoing mitosis is the cdc25 phosphatase, an important regulator of cdc2 activity (Crenshaw, et al. The mitotic peptidyl-prolyl isomerase, Pin1, interacts with Cdc25 and Plx1. EMBO J 17:1315-1327, 1998; Shen, et al. The essential mitotic peptidyl-prolyl isomerase Pin1 binds and regulates mitosis-specific phosphoproteins. Genes & Development 12:706-720, 1998). Cdc25 is responsible for the activation of cdc2 by removal of an inhibitory phosphorylation on tyrosine 15. Cdc25 is itself activated by phosphorylation, and several phosphorylation sites have been identified (i.e., at threonine 48) (Strausfeld, et al. Activation of p34cdc2 protein kinase by microinjection of human cdc25C into mammalian cells. Requirement for prior phosphorylation of cdc25C by p34cdc2 on sites phosphorylated at mitosis. J Biol Chem 269:5989-6000, 1994; Izumi, et al. Blimination of cdc2 phosphorylation sites in the cdc25 phosphatase blocks initiation of M-phase. Mol Biol Cell 4:1337-1350, 1993). At one or more of these, Pin1 binding alters the conformation of and prevents the activation of cdc25, thus delaying activation of cdc2.

In normal mitotic cells, this inhibition is transient, as the conformational changes in cdc25 are reversible. The action of Pin1 on cdc25 during mitosis is thus to regulate the timing of activation of this phosphatase, and hence the precise timing of the action of cdc2. Disruption of this timing is catastrophic for the cells undergoing mitosis, and results in apoptosis or death through other less well-characterized mechanisms (Shuster, et al. Parameters that specify the timing of cytokinesis. 3 Cell Biol 146:981-992, 1999; Creanor, et al. The kinetics of the B cyclin p56cdc13 and the phosphatase p80cdc25 during the cell cycle of the fission yeast Schizosaccharomyces pombe. J Cell Sci 109:1647-1653, 1996.).

Interestingly, binding of Pin1 to cdc2-phosphorylated tau was demonstrated to alter the conformation of the protein. Further work showed that Pin1 was tightly associated with phosphorylated tau isolated from the AD brain, and was co-localized with neurofibrillary tangles in sections of AD brain tissue (Lu, et al. The prolyl isomerase Pin1 restores the biological function of Alzheimer-associated phosphorylated tau. Nature, 399, 784-788, 1999). Binding of Pin1 to phosphorylated tau has also been demonstrated to alter the conformation of tau, and it is likely that this also occurs on binding of Pin1 to other proteins (Shen et al. The essential mitotic peptidyl-prolyl isomerase Pin1 binds and regulates mitosis-specific phosphoproteins. Genes Dev. 12, 706-720. 1998). While low levels of phosphorlation of tau at this site probably do occur in the normal brain, the extent of phosphorylation of threonine 231 is greatly increased in Alzheimer's disease brains. It is possible to propose that phosphorylation of tau by cdc2 at thr231 leads to the binding of Pin1, and that this protein alters the conformation of tau such that it forms PHF, which then aggregate into neurofibrillary tangles. Cell death could result either from the abnormalities of tau, or from depletion and sequestration of Pin1 into the tangles. This scheme provides an explanation linking both phosphorylation and conformational changes of tau, both of which are established as early events in the neuronal abnormalities of AD.

Binding agents that prevent the interaction of proteins with the C-terminal region of APP containing phosphothreonine 668 are expected to have significant effects on the proteolytic processing of APP and production of the 40 to 42 amino acid peptide. Such binding agents are likely to be useful in slowing the rate of development of AD pathology. Given the structural similarities between tau and APP. surrounding thr231 and thr668, respectively, it is likely that single binding agents capable of interacting with either site may be identified. Such binding agents would have a major advantage over currently available forms of therapy for AD. However, such binding agents are not currently available.

Thus, there exists a need in the art for binding agents (i.e., compounds) that interfere with the formation of complexes between tau, APP, edc25, for example, and their respective binding partners. Such binding agents are useful for inhibiting the development or progression of AD and/or cancer, for example. An exemplary system is provided herein as reagents and methods for identifying binding agents that interfere with the interaction of Pin 1 and targets of Pin 1, such as tau and APP, thus blocking the access of Pin 1 or other proteins to those target proteins. Such blockage is expected to prevent the formation of abnormal conditions, such as disease-realted conformations of tau, abnormal proteolytic processing of APP, and pathology in AD.

### SUMMARY OF THE INVENTION

The invention provides reagents and methodologies for identification and isolation of binding agents useful for preventing or treating diseases such as Alzheimer's Disease (AD) and cancer, for example. The binding agents are capable of interfering with the interaction of at least two proteins, polypeptides, peptides, fragments thereof and/or derivatives thereof. Typically, at least a first and a second protein, polypeptide, peptide, fragment thereof or derivative thereof interact with one another (i.e., bind and thereby affect the function of one or the other, or both), and contribute to the pathology of AD or cancer, for example. The invention provides reagents and methodologies for identifying binding agents (i.e., compounds) that interfere with such interactions.

In one embodiment, the proteins, polypeptides, peptides, fragment thereof, or derivative thereof may be represented by a binding surrogate. High-throughput systems are provided in which a binding surrogate corresponding to a binding partner (i.e., a polypeptide known to interact with a peptide) of a peptide is utilized in the assay. The binding surrogate may be any protein, polypeptide, peptide, fragment thereof, derivative thereof or any other compound that at least substantially retains or mimics the function of a protein. Interference of the interaction between the binding surrogate and the peptide by the binding agent is determined. A binding agent that interferes with the interaction of a peptide and a binding surrogate is selected as a “desired” binding agent. Such binding agents may be further developed for use in diagnostics, prevention and treatment of disease. In certain embodiments, the polypeptide per se may be referred to as a binding surrogate.

In one embodiment, a test binding agent is added to a reaction mixture comprising a peptide and a binding surrogate. In another embodiment, a test binding agent, peptide and the binding surrogate are concurrently added to a reaction mixture. In yet another embodiment, the binding surrogate and the binding agent are first reacted, and the peptide is then added to the reaction mixture. In any of these embodiments, the effect of the test binding agent on the interaction between the peptide and the binding surrogate is measured. Inhibition of the interaction, as demonstrated by decreased binding between the peptide and the binding surrogate, indicates that the test binding agent is a desired binding agent. It should be understood that these methods are suitable whether a protein, polypeptide, peptide, fragment thereof or derivative thereof is utilized.

In one embodiment, the invention provides a method for identifying a desired binding agent that interferes with the interaction between a peptide and a binding surrogate by combining the peptide, the binding surrogate, and a test binding agent to form a reaction mixture; detecting binding between the peptide and the binding surrogate; wherein a decrease in the interaction between the peptide and the binding surrogate indicates that the test binding agent interferes with the interaction. Another aspect of the invention relates to the inclusion of a control reaction in which the level of interaction between the peptide and the binding surrogate in the absence of the test binding agent is determined as a control and compared to the level of interaction detected. A desired binding agent results in a decreased level of interaction as compared to the level of interaction detected in the control reaction. As previously mentioned, a reactive polypeptide may be utilized in place of the binding surrogate. Other non-limiting embodiments will become apparent from the descriptions provided below.

### BRIEF DESCRIPTION OF THE DRAWINGS

FIG. 1. Binding curves showing the interaction of the monoclonal antibody MC2 to a tau peptide incorporating the phosphorylated threonine 231 site (tau231P) and an APP peptide incorporating the phosphorylated threonine 668 (APP668P) site. MC2 does not bind to the respective non-phosphorylated peptides.

FIG. 2. Binding curves showing Pin1 interactions with tau231P and APP668P. Pin1 does not bind to the respective non-phosphorylated peptides.

FIG. 3. Antibodies reactive with APP668P. A. Antibody GF10. B. Antibody GF11. C. Antibody GF20. D. Antibody GP27.

FIG. 4. Antibodies reactive with tau231P and APP668P. A. Antibody GF3. B. Antibody GF5.

FIG. 5. A. GF31 binding to tau and APP phosphopeptides. B. Inhibition of GF31 binding to tau231P by TG3.

FIG. 6. GF31 inhibition of Pin1 binding to APP668P.

### DETAILED DESCRIPTION

As used herein, “thr231” refers to the threonine 231 of tau, “thr668” refers to the threonine 668 of APP and “thr48” refers to threonine 48 of cdc25. Further, “th231P” refers to phosphorylated thr231, “thr688P” refers to phosphorylated thr668 and thr48P refers to phosphorylated thr48. Still further, “tau231P” refers to a tau polypeptide comprising thr231P “APP668P” refers to a APP polypeptide comprising thr668P and cdc2548P refers to a cdc25 polypeptide comprising thr48P. The terms tau231P, APP668P and cdc25-48P may be refer to a full-length, fragment or derivative of phosphorylated tau, phosphorylated APP, or phosphorylated cdc25, respectively.

As used herein, the term “binding agent” refers to a compound or molecule having binding specificity for one or more proteins (or a polypeptide, peptide, fragment and/or derivative corresponding to the protein) that are involved in a disease process. In a preferred embodiment, the suitable binding agent is a compound inhibits the binding of one protein to another protein. Suitable binding agents include, but are not limited to, antibodies and derivatives thereof, peptides, polypeptides, proteins and small molecules such as organic molecules of less than about 1000 g/mol. Suitable binding agents may be prepared using methods known in the art. The binding agents are useful for heating disorders related to the interaction of proteins.

Disorders that may be diagnosed or treated include but are not limited to cancer and Alzheimer's Disease (AD), for example. Cancer is defined herein as any cellular malignancy for which a loss of normal cellular controls results in unregulated growth, lack of differentiation, and increased ability to invade local tissues and metastasize. Cancer may develop in any tissue of any organ at any age. Cancer may be an inherited disorder or caused by environmental factors or infectious agents; it may also result from a combination of these. For the purposes of utilizing the invention, the term cancer includes both neoplasms and premalignant cells.

In one embodiment, the invention relates to reagents and methods for discovering binding agents that interfere with the interaction between proteins such as tau, APP, and/or cdc25 and their respective binding partners (i.e., tau and Pin 1). In another embodiment, the invention relates to the identification of binding agents in the form of compounds that disrupt or inhibit the interaction of one protein with another. In one embodiment of the invention, the binding agent interferes with the interaction of Pin 1 with tau and /or APP, proteins known to be associated with AD. In another embodiment, Pin 1 may interact with proteins related to the development or progression of cancer, such as cdc25.. Binding agents identified using the instant methodology may be utilized to interrupt the interaction of Pin 1 with such proteins, thereby preventing or inhibiting AD or cancer progression. It should understood by the skilled artisan that the methodologies described herein are applicable to many different proteins and that reference to Pin1 is merely exemplary and non-limiting.

Binding agents such as antibodies and antibody fragments that bind a protein, protein fragment or peptide of the invention are within the scope of the invention. The antibodies may be polyclonal including monospecific polyclonal; monoclonal (mAbs); recombinant; chimeric; humanized, such as CDR-grafted; human; single chain; and/or bispecific; as well as fragments; variants; or derivatives thereof Antibody fragments include those portions of the antibody that bind to an epitope on the protein, protein fragment or peptide of the invention. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Exemplary antibody molecules for use in the diagnostic methods and systems of the invention are intact immunoglobulin molecules, substantially intact immunoglobulin molecules and those portions of an immunoglobulin molecule that contain the paratope, including those portions known in the art as Fab, Fab′, F(ab′)₂ and F(v). Fab and F(ab′)₂ portions of antibodies are prepared by the proteolytic reaction of papain and pepsin, respectively, on substantially intact antibodies by methods that are well known. (See for example, U.S. Pat. No. 4,342,566 to Theofilopolous and Dixon.) Fab′ antibody portions are also well known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules are preferred, and are utilized as illustrative herein.

The preparation of antibodies against a polypeptide is well known in the art. (See Staudt et al., J. Exp. Med., 157:687-704 (1983), or the teachings of Sutcliffe, J. G., as described in U.S. Pat. No. 4,900,811, the teaching of which are hereby incorporated by reference.) Briefly, to produce an antibody composition of this invention, a laboratory mammal is inoculated with an immunologically effective amount of a polypeptide of this invention. The anti-polypeptide antibody molecules thereby induced are then collected from the mammal and those immunospecific for both a polypeptide and the corresponding recombinant protein are isolated to the extent desired by well known techniques such as, for example, by immunoaffinity chromatography.

To enhance the specificity of the antibody, the antibodies are preferably purified by immunoaffinity chromatography using solid phase-affixed immunizing polypeptide. The antibody is contacted with the solid phase-affixed immunizing polypeptide for a period of time sufficient for the polypeptide to immunoreact with the antibody molecules to form a solid phase-affixed immunocomplex. The bound antibodies are separated from the complex by standard techniques.

For a polypeptide that contains fewer than about 35 amino acid residues, it is preferable to use the peptide bound to a carrier for the purpose of inducing the production of antibodies. One or more additional amino acid residues can be added to the amino- or carboxy-termini of the polypeptide to assist in binding the polypeptide to a carrier. Cysteine residues added at the amino- or carboxy-termini of the polypeptide have been found to be particularly useful for forming conjugates via disulfide bonds. However, other methods well known in the art for preparing conjugates can also be used. The techniques of polypeptide conjugation or coupling through activated functional groups presently known in the art are particularly applicable. See, for example, Aurameas, et al., Scand. J. Immunol., Vol. 8, Suppl. 7:7-23 (1978) and U.S. Pat. No. 4,493,795, No. 3,791,932 and No. 3,839,153. In addition, a site directed coupling reaction can be carried out so that any loss of activity due to polypeptide orientation after coupling can be minimized. See, for example, Rodwell et al., Biotech., 3:889-894 (1985), and U.S. Pat. No. 4,671,958. Exemplary additional linking procedures include the use of Michael addition reaction products, di-aldehydes such as glutaraldehyde, Klipstein, et al., J. Infect. Dis., 147:318-326 (1983) and the like, or the use of carbodiimide technology as in the use of a water-soluble carbodiimide to form amide links to the carrier. Alternatively, the heterobifunctional cross-linker SPDP (N-succinimidyl-3-(2-pyridyldithio) proprionate)) can be used to conjugate peptides, in which a carboxy-terminal cysteine has been introduced.

Useful carriers are well known in the art, and are generally proteins themselves. Exemplary of such carriers are keyhole limpet hemocyanin (KLH, edestin, thyroglobulin, albumins such as bovine serum albumin (3SA) or human serum albumin (HSA), red blood cells such as sheep erythrocytes (SRBC), tetanus toxoid, cholera toxoid as well as polyamino acids such as poly D-lysine:D-glutamic acid, and the like. The choice of carrier is more dependent upon the ultimate use of the inoculum and is based upon criteria not particularly involved in the invention. For example, a carrier that does not generate an untoward reaction in the particular animal to be inoculated should be selected.

The present inoculum contains an effective, immunogenic amount of a polypeptide, typically as a conjugate linked to a carrier. The effective amount of polypeptide per unit dose sufficient to induce an immune response to the immunizing polypeptide depends, among other things, on the species of animal inoculated, the body weight of the animal and the chosen inoculation regimen is well known in the art. Inocula typically contain polypeptide concentrations of about 10 micrograms (μg) to about 500 milligrams (mg) per inoculation (dose), preferably about 50 micrograms to about 50 milligrams per dose. The term “unit dose” as it pertains to the inocula refers to physically discrete units suitable as unitary dosages for animals, each unit containing a predetermined quantity of active material calculated to produce the desired immunogenic effect in association with the required diluent; i.e., carrier, or vehicle. The specifications for the novel unit dose of an inoculum of this invention are dictated by and are directly dependent on (a) the unique characteristics of the active material and the particular immunologic effect to be achieved, and (b) the limitations inherent in the art of compounding such active material for immunologic use in animals, as disclosed in detail herein, these being features of the invention.

Inocula are typically prepared from the dried solid polypeptide-conjugate by dispersing the polypeptide-conjugate in a physiologically tolerable (acceptable) diluent such as water, saline or phosphate-buffered saline to form an aqueous composition. Inocula can also include an adjuvant as part of the diluent. Adjuvants such as complete Freund's adjuvant (CFA), incomplete Freund's adjuvant (IFA) and alum are materials well known in the art, and are available commercially from several sources.

The antibody so produced can be used, inter alia, in the methods and systems of the invention to detect a polypeptide in a sample such as a tissue section or body fluid sample. Anti-polypeptide antibodies that inhibit function of the polypeptide can also be used in vivo in therapeutic methods as described herein. A preferred anti-polypeptide antibody is a monoclonal antibody. The phrase “monoclonal antibody” in its various grammatical forms refers to a population of antibody molecules that contain only one species of antibody combining site capable of immunoreacting with a particular epitope. A monoclonal antibody thus typically displays a single binding affinity for any epitope with which it immunoreacts. A monoclonal antibody may therefore contain an antibody molecule having a plurality of antibody combining sites, each immunospecific for a different epitope, e.g., a bispecific monoclonal antibody. A preferred monoclonal antibody of this invention comprises antibody molecules that immnunoreact with a polypeptide of the invention. More preferably, the monoclonal antibody also immnunoreacts with recombinantly produced whole protein.

A monoclonal antibody is typically composed of antibodies produced by clones of a single cell called a hybridoma that secretes (produces) only one kind of antibody molecule. The hybridoma cell is formed by fusing an antibody-producing cell and a myeloma or other self-perpetuating cell line. The preparation of such antibodies was first described by Kohler and Milstein, Nature, 256:495-497 (1975), the description of which is incorporated by reference. The hybridoma supemates so prepared can be screened for the presence of antibody molecules that immunoreact with a polypeptide.

Briefly, to form the hybridoma from which the monoclonal antibody composition is produced, a myeloma or other self-perpetuating cell line is fused with lymphocytes obtained from the spleen of a mammal hyperimmiunized with a antigen, such as is present in a polypeptide described herein. The polypeptide-induced hybridoma technology is described by Niman et al., Proc. Natl. Acad. Sci., USA, 80:4949-4953 (1983), the description of which is incorporated herein by reference. It is preferred that the myeloma cell line used to prepare a hybridoma be from the same species as the lymphocytes. Typically, a mouse of the strain 129 GIX⁺ is the preferred mammal. Suitable mouse myelomas for use in the invention include the hypoxanthine-aminopterin-thymidine-sensitive (HAT) cell lines P3X63-Ag8.653, and Sp2/0-Ag14 that are available from the American Type Culture Collection, Rockville, Md., under the designations CRL 1580 and CRL 1581, respectively. Splenocytes are typically fused with myeloma cells using polyethylene glycol (PEG) 1500. Fused hybrids are selected by their sensitivity to HAT. Hybridomas producing a monoclonal antibody of this invention are identified using the enzyme linked immunosorbent assay (ELISA).

A monoclonal antibody of the invention can also be produced by initiating a monoclonal hybridoma culture comprising a nutrient medium containing a hybridoma that produces and secretes antibody molecules of the appropriate polypeptide specificity. The culture is maintained under conditions and for a time period sufficient for the hybridoma to secrete the antibody molecules into the medium. The antibody-containing medium is then collected. The antibody molecules can then be ether isolated by well known techniques. Media useful for the preparation of these compositions are both well known in the art and commercially available and include synthetic culture media, inbred mice and the like. An exemplary synthetic medium is Dulbecco's Minimal Essential Medium (DMEM; Dulbecco et al., Virol. 8:396 (1959)) supplemented with 4.5 gm/1 glucose, 20 mM glutamine, and 20% fetal calf serum. An exemplary inbred mouse strain is the Balb/c. Other methods of producing a monoclonal antibody, a hybridoma cell, or a hybridoma cell culture are also well known (See, for example, The method of isolating monoclonal antibodies from an immunological repertoire, as described by Sastry, et al., Proc. Natl. Acad. Sci. USA, 86:5728-5732 (1989); and Huse et al., Science, 246:1275-1281 (1989)).

The monoclonal antibodies of this invention can be used in the same manner as disclosed herein for antibodies of the invention. For example, the monoclonal antibody can be used in the therapeutic, diagnostic or in vitro methods disclosed herein where immunoreaction with a polypeptide of the invention is desired. Also contemplated by this invention is the hybridoma cell and cultures containing a hybridoma cell that produce a monoclonal antibody of this invention.

It is also possible to isolate antibodies reactive against polypeptides of the invention using phage display techniques. Display of antibody fragments on the surface of viruses which infect bacteria (bacteriophage or phage) makes it possible to produce human sFvs with a wide range of affinities and kinetic characteristics. To display antibody fragments on the surface of phage (phage display), an antibody fragment gene is inserted into the gene encoding a phage surface protein (pIII) and the antibody fragment-pIII fusion protein is expressed on the phage surface (McCafferty et al. (1990) Nature, 348: 552-554; Hoogenboom et al. (1991) Nucleic Acids Res., 19: 4133-4137). For example, a sFv gene coding for the V_{H} and V_{L} domains of an anti-lysozyme antibody (D1.3) was inserted into the phage gene III resulting in the production of phage with the DI.3 sFv joined to the N-terminus of pIII thereby producing a “fusion” phage capable of binding lysozyme (McCafferty et al (1990) Nature, 348: 552-554). The skilled artisan may also refer to Clackson et al. (1991) Nature, 352: 624-628), (Marks et al. (1992) Bio/Technology, 10: 779-783), Marks et al Bio/Technology, 10: 779-785 (1992) for further guidance. In the present case, the antibody fragment gene is isolated from the immunized mammal, and inserted into the phage display system. Phage containing antibodies reactive to the polypeptide are then isolated and characterized using well-known techniques. Kits and services are available for generating antibodies by phage display from well-known sources such as Cambridge Antibody Technology Group plc (United Kingdom).

Another embodiment is a “chimeric” antibody in which a portion of the heavy and/or light chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. Also included are fragments of such antibodies, so long as they exhibit the desired biological activity. See U.S. Pat. No. 4,816,567; Morrison et al., 1985, Proc. Natl. Acad. Sci. 81:6851-55.

In another embodiment, a monoclonal antibody of the invention is a “humanized” antibody. Methods for humanizing non-human antibodies are well known in the art. See U.S. Pat. Nos. 5,585,089 and 5,693,762. Generally, a humanized antibody has one or more amino acid residues introduced into it from a source that is non-human. Humanization can be performed, for example, using methods described in the art (Jones et al., 1986, Nature 321:522-25; Riechmann et al., 1998, Nature 332:323-27; Verhoeyen et al., 1988, Science 239:1534-36), by substituting at least a portion of a rodent complementarity-determining region (CDR) for the corresponding regions of a human antibody.

Also encompassed by the invention are human antibodies that bind polypeptides. Using transgenic animals (e.g., mice) that are capable of producing a repertoire of human antibodies in the absence of endogenous immunoglobulin production such antibodies are produced by immunization with a polypeptide antigen (i.e., having at least 6 contiguous amino acids), optionally conjugated to a carrier. See, e.g., Jakobovits et al., 1993, Proc. Natl. Acad. Sci. 90:2551-55; Jakobovits et al., 1993, Nature 362:255-58; Bruggermann et al., 1993, Year in Immuno. 7:33. In one method, such transgenic animals are produced by incapacitating the endogenous loci encoding the heavy and light immunoglobulin chains therein, and inserting loci encoding human heavy and light chain proteins into the genome thereof Partially modified animals, that is those having less than the full complement of modifications, are then crossbred to obtain an animal having all of the desired immune system modifications. When administered an immunogen, these transgenic animals produce antibodies with human (rather than, e.g., murine) amino acid sequences, including variable regions which are immunospecific for these antigens. See PCT App. Nos. PCT/US96/05928 and PCT/US93/06926. Additional methods are described in U.S. Pat. No. 5,545,807, PCT App. Nos. PCT/US91/245 and PCT/GB89/01207, and in EP Pub. Nos. 546073B1 and 546073A1. Human antibodies can also be produced by the expression of recombinant DNA in host cells or by expression in hybridoma cells as described herein.

In an alternative embodiment, human antibodies can also be produced from phage-display libraries (Hoogenboom et al., 1991, J. Mol. Biol. 227:381; Marks et al, 1991, J. Mol. Biol. 222:581). These processes mimic immune selection through the display of antibody repertoires on the surface of filamentous bacteriophage, and subsequent selection of phage by their binding to an antigen of choice. One such technique is described in PCT App. No. PCT/US98/17364, which describes the isolation of high affinity and functional agonistic antibodies for MPL- and msk-receptors using such an approach.

Chimeric, CDR grafted, and humanized antibodies are typically produced by recombinant methods. Nucleic acids encoding the antibodies are introduced into host cells and expressed using materials and procedures described herein. In a preferred embodiment, the antibodies are produced in mammalian host cells, such as CHO cells. Monoclonal (e.g., human) antibodies may be produced by the expression of recombinant DNA in host cells or by expression in hybridoma cells as described herein.

The anti-polypeptide antibodies of the invention may be employed in any known assay method, such as competitive binding assays, direct and indirect sandwich assays, and immunoprecipitation assays (Sola, Monoclonal Antibodies: A Manual of Techniques 147-158 (CRC Press, Inc., 1987)) for the detection and quantitation of polypeptides. The antibodies will bind the polypeptides with an affinity that is appropriate for the assay method being employed.

Binding agents of the invention may be used as therapeutics. These therrpeutic agents are generally agonists or antagonists, in that they either enhance or reduce, respectively, at least one of the biological activities of a polypeptide. In one embodiment, antagonist antibodies of the invention are antibodies or binding fragments thereof which are capable of specifically binding to a polypeptide and which are capable of inhibiting or eliminating the functional activity of a polypeptide in vivo or in vitro. In preferred embodiments, the binding agent, e.g., an antagonist antibody, will inhibit the functional activity of a polypeptide by at least about 50%, and preferably by at least about 80%. In another embodiment, the binding agent may be an anti-polypeptide antibody that is capable of interacting with a polypeptide binding partner (a ligand or receptor) thereby inhibiting or eliminating polypeptide activity in vitro or in vivo. Binding agents, including agonist and antagonist anti-polypeptide antibodies, are identified by screening assays that are well known in the art.

As described above, a binding agent may comprise an antibody molecule. An antibody of the invention is typically produced by immunizing a mammal with an inoculum containing a protein, protein fragment, or peptide of this invention (i.e., tau, APP, tau231P, APP668P), collectively referred to as polypeptide, and thereby induce in the mammal antibody molecules having immunospecificity for immunizing polypeptide. The antibody molecules are then collected from the mammal and isolated to the extent desired by well-known techniques such as, for example, by using DEAB Sephadex or Protein G to obtain the IgG fraction.

Exemplary antibodies capable of binding to tau231P, APP668P, and/or phosphopeptides representative thereof are provided herein. For example, AT180, TG3, CP9, CP10, CP16, CP17, GF1, GF7, GF25, MC2, GF10, GF11, GF20, GP27, GF3, GF5, GF31, and a polyclonal antibody (Suzuki, et al. Cell cycle-dependent regulation of the phosphorylation and metabolism of the Alzheimer amyloid precursor protein EMBO J. 13:1114-1122, 1994) are provided herein or have been previously described in the field. These antibodies are useful for binding tau231P, APP668P, and/or phosphopeptides representative thereof. In particular, MC2 binds to both phosphorylated tau and APP. GF10, GF11, GF20, and GF27 preferably bind to APP668P. Antibodies GF3 and GF5 preferably bind to tau231P. Gk31 is capable of binding to peptides corresponding to either tau231P or APP668P. Many other antibodies would be suitable for binding to tau231P, APP668P, and /or phosphopeptides representative thereof and would be useful in practicing the invention. Such antibody molecules are encompassed by the invention.

In one embodiment, the invention provides a method for identifying suitable binding agents. For example, the invention provides a method for combining a protein, polypeptide, peptide, fragment thereof, or derivative thereof; a second protein, polypeptide, peptide, fragment thereof, or derivative thereof (i.e., a protein that interacts with the first protein or suitable binding surrogate therefor); a test binding agent; and detecting the interaction of the first and second, protein, polypeptide, peptide, fragment thereof, or derivative thereof to determine whether the test binding agent interferes with the interaction.

In one embodiment, the components are combined into a single reaction mixture under conditions in which the first and second protein, protein fragment or peptide would interact (i.e., bind to one another). A control sample may be utilized that does not contain the test-binding agent. Another sample may be prepared that contains the first and second proteins, protein fragments or peptides along with the test binding agent. Inhibition of binding of the second protein, polypeptide, peptide, fragment thereof, or derivative thereof to the first protein, protein fragment or peptide by the test binding agent indicates that the binding agent qualifies for further study. The level of binding in the experimental sample may be compared to that of the control sample to determine whether or not the interaction has been inhibited, enhanced, or not affected. In one embodiment, inhibition of binding of the second protein, protein fragment or peptide to the first protein, protein fragment or peptide by the test binding agent indicates that the binding agent qualifies for further study. In another embodiment, enhancement of binding of the second protein, protein fragment or peptide to the first protein, protein fragment or peptide by the test binding agent indicates that the binding agent qualifies for further study. Various reaction conditions may be tested to determine the effects of the test binding agent on the interaction of the first and second proteins, protein fragments or peptides. In addition, any combination of first and second proteins, protein fragments or peptides may be utilized in practicing the invention. For example, a first polypeptide member of the reaction may be a protein while the second polypeptide member of the reaction is a peptide. Other variations of these embodiments would be understood by those of skill in the art.

In one embodiment, the assay utilizes a protein, protein fragment or peptide as the substrate to which another polypeptide or binding surrogate binds. In one embodiment, a peptide having amino acid sequence similar to at least a portion of tau, APP, and/or cdc25 is utilized. The amino acid similarities may reside in multiple peptides (i.e., peptide A having similarity to tau, peptide B having similarity to APP, and peptide C having similarity to cdc25) or a single peptide having regions of identity with one or more of tau, APP and/or cdc25. In a preferred embodiment, the peptide comprises at least thr231 of tau (“thr231”), thr668 of APP (“thr668”), thr48 of cdc25 (“thr48”). In a more preferred embodiment, the peptide or peptides comprises thr231 and/or thr668 and/or thr48, along with the naturally occurring amino acid residues that surround these sites. In an even more preferred embodiment, the peptides are phosphorylated at the amino acids corresponding to thr231 and/or thr668 and/or thr48. Exemplary peptides include but are not limited to biotin-KKVAVVR(phospho)TPPKSPSS (SEQ ID NO. 1) (corresponding tau231P), biotin-VEVDAAV(phospho)TPEERHLS (SEQ ID NO. 2) (corresponding to APP668P), or biotin-VCPDVPR(phospho)TPVGFLG (SEQ ID NO. 3) (corresponding to cdc25-48P). Any suitable protein, protein fragment or peptide may be utilized, as would be understood by one of skill in the art.

A second protein, protein fragment, polypeptide or peptide is also utilized in practicing the invention. It is preferred that the second protein, protein fragment, polypeptide or peptide interact with the first protein, protein fragment, polypeptide or peptide. It is further preferred that the second protein, protein fragment, polypeptide, peptide, fragment thereof or derivative thereof interact with the first protein, protein fragment or peptide in a modified form (i.e., phosphorylated, sulfated, glycosylated, etc.), as would be found in vivo. The modifications may occur following translation of such a first protein, polypeptide, peptide, fragment thereof or derivative thereof in, for example, a recombinant expression system, or using in vitro techniques following synthetic production of the first protein, polypeptide, peptide, fragment thereof or derivative thereof. Such modifications are known in the art and are encompassed by the invention.

In a preferred embodiment, where the peptide is derived from tau, APP or cdc25, interaction of the protein with the peptide is related to thr231 and /or thr668 and/or thr48. In a more preferred embodiment, the protein interacts with tau231P and /or APP668P and/or cdc25-48P. An exemplary protein for use in the instant methodology is Pin 1(see, for example, the coding sequence of ATCC #555784).

An exemplary binding surrogate is a compound, such as a peptide, corresponds to the second protein, such as Pin 1, for example. By “corresponds to” is meant that the binding surrogate binds to the first protein, protein fragment, polypeptide or peptide at the same or similar site as Pin 1, for example. The binding surrogate may or may not have the same sequence as the protein to which it corresponds. For example, the binding surrogate may share sequence identity with Pin 1, or may be of a different sequence but have the same or similar binding activity. The binding surrogate, then, is capable of forming the same or a similar interaction with the first protein, protein fragment, polypeptide or peptide as is the second protein (i.e., Pin 1). In certain embodiments, the binding surrogate may also be the second protein itself (i.e., Pin 1), or a fragment or derivative thereof. In other embodiments, the binding surrogate may be an antibody, such as a monoclonal antibody or polyclonal antisera. Other suitable binding surrogates would be understood by one of skill in the art.

As described above, Pin1 is also known to interact with tau and APP. It is known, for example, that Pin1 binds to the phosphorylated threonine 231 of tau. In addition, it is known that Pin1 binds to phosphopeptides derived from tau as well as APP. The invention provides reagents and methods for identifying binding agents that interfere with the interaction of proteins such as Pin1 with proteins such as tau and APP. As the interaction of proteins with tau and APP has been correlated with AD, prevention or interference with such interactions is desired by those with the disease. An exemplary binding agent is the antibody GF3, which binds to peptides corresponding to either tau231P or APP668P and interferes with the binding of Pin1 to these peptides. Other suitable binding agents are also encompassed by the invention.

Binding agents that prevent the interaction of proteins with the C-terminal region of APP containing thr668P are expected to have significant effects on the proteolytic processing of APP, and hence on production of the 40 to 42 amino acid peptide. Such binding agents are likely to be useful in slowing the rate of development of Alzheimer's disease pathology. Given the structural similarities between tau and APP in the area of the appropriate threonines, it is likely that binding agents that interact with both sites may be identified. Such binding agents would have a major advantage over any other potential form of therapy for AD, as the binding agents would be expected to prevent or slow the development of abnormal pathologic structures (plaques and tangles) by influencing both of the major proteins involved in the formation of such structures.

The methodologies provided herein also relate to identification of binding agents that interfere with the function of cdc25.

In one embodiment, the invention relates to binding agents that affect these pathways. Binding agents that inhibit the binding of Pin 1 with cdc25 are desirable. The Pin 1 protein itself or a suitable binding surrogate may be utilized to identify such binding agents. For example, Pin 1 and cdc25 may be incubated under conditions suitable for interaction between the proteins to take place. A test binding agent may then be added, and the effect of the test binding agent on the interaction of Pin 1 with cdc25 measured. The interaction may be measured by detecting, for example, the amount of Pin 1 bound to cdc25 in the presence or absence of the test binding agent. A decrease in the amount of Pin 1 bound to cdc25 following exposure to the test binding agent indicates that the binding agent is desirable and useful for blocking the interaction of Pin 1 and cdc25. In other embodiments, a binding surrogate such as a peptide that corresponds to the sequence or binding activity of Pin 1 may be utilized. A suitable peptide would be one having a Pin 1 binding domain or a domain that mimics the binding of Pin 1, such as a monoclonal antibody or derivative thereof (i.e, F_{ab} fragment). Any such peptide would be suitable provided the peptide interacted with cdc25 in approximately the same manner as Pin 1. Other variations of this assay would be understood by those of skill in the art.

In one embodiment, the first protein, protein fragment, polypeptide or peptide is labeled with biotin or other suitable label. For example, a peptide comprising tau231P, APP668P, or cdc2548P may be labeled with biotin using standard techniques. The biotinylated peptide is then attached to an avidin-coated reaction vessel, such as a 96-well plate. The first protein, polypeptide, peptide, fragment thereof or derivative thereof (i.e., tau231P, APP668P, or cdc2548P) may be modified, such as by phosphorylation, either as isolated or in vitro using standard techniques (Jicha, et al. Conformation and phosphorylation dependent antibody recognizing the paired helical filaments of Alzheimer's Disease. J. Neurochem. 69, 2087-2095, 1997). Other suitable labels and labeling techniques may be suitable and are known in the art.

The reactions preferably take place within a suitable container such as a microtiter plate. Preferably, the plate has at least 96 wells, but plates with more wells, such as 384 or 1526 wells, are also suitable for large screening assays. The plate is preferably constructed of a suitably solid and inert material such as polystyrene or polypropylene. Prior to using the plate for practicing the invention, the plate may be coated with a material such as avidin, streptavidin or the like. This is particularly useful where the first protein has been labeled with, for example, biotin.

For use in diagnosis, prevention or treatment of a disease such as AD or cancer, the binding agents of the invention may be administered orally, parentally, by inhalation spray, rectally, or topically in dosage unit formulations containing conventional pharmaceutically acceptable carriers, adjuvants, and vehicles. The term parenteral as used herein includes, subcutaneous, intravenous, intramuscular, intrasternal, infusion techniques or intraperitoneally. Suppositories for rectal administration of the drug can be prepared by mixing the drug with a suitable non-irritating excipient such as cocoa butter and polyethylene glycols that are solid at ordinary temperatures but liquid at the rectal temperature and will therefore melt in the rectum and release the drug.

The dosage regimen for treating a neurological disorder disease with the binding agents of this invention and/or compositions of this invention. is based on a variety of factors, including the type of disease, the age, weight, sex, medical condition of the patient, the severity of the condition, the route of administration, and the particular compound employed. Thus, the dosage regimen may vary widely, but can be determined routinely using standard methods.

The pharmaceutically active binding agents (i.e., compounds) of this invention can be processed in accordance with conventional methods of pharmacy to produce medicinal agents for administration to patients, including humans and other mammals. For oral administration, the pharmaceutical composition may be in the form of, for example, a capsule, a tablet, a suspension, or liquid. The pharmaceutical composition is preferably made in the form of a dosage unit containing a given amount of binding agent. A suitable daily dose for a human or other mammal may vary widely depending on the condition of the patient and other factors, but, once again, can be determined using routine methods. The binding agent may also be administered by injection as a composition with suitable carriers including saline, dextrose, or water.

Injectable preparations, such as sterile injectable aqueous or oleaginous suspensions, may be formulated according to the known are using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

A suitable topical dose of active ingredient of a binding agent of the invention is administered one to four, preferably two or three times daily. For topical administration, the binding agent may comprise from 0.001% to 10% w/w, e.g., from 1% to 2% by weight of the formulation, although it may comprise as much as 10% w/w, but preferably not more than 5% w/w, and more preferably from 0.1% to 1% of the formulation. Formulations suitable for topical administration include liquid or semi-liquid preparations suitable for penetration through the skin (e.g., liniments, lotions, ointments, creams, or pastes) and drops suitable for administration to the eye, ear, or nose.

The pharmaceutical compositions may be made up in a solid form (including granules, powders or suppositories) or in a liquid form (e.g., solutions, suspensions, or emulsions). The pharmaceutical compositions may be subjected to conventional pharmaceutical operations such as sterilization and/or may contain conventional adjuvants, such as preservatives, stabilizers, wetting agents, emulsifiers, buffers etc. Solid dosage forms for oral administration may include capsules, tablets, pills, powders, and granules. In such solid dosage forms, the active compound may be admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings. Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting sweetening, flavoring, and perfuming agents.

While the binding agents of the invention can be administered as the sole active pharmaceutical agent, they can also be used in combination with one or more compounds of the invention or other agents. When administered as a combination, the therapeutic agents can be formulated as separate compositions that are given at the same time or different times, or the therapeutic agents can be given as a single composition.

### EXAMPLES

### Example 1

### Antibodies that Bind Phosphothreonine 231 of Tau (Tau231P) and/or Phosphothreonine 668 of APP (APP668P)

A. Materials and Methods

To identify compounds which bind to tau peptides phosphorylated on threonine 231, a 96 well or 386 well ELISA plate is coated with Neuravidin in 20 mM K₂HPO₄/10 mM KH₂PO₄, 1 mM EDTA, 0.8% NaCl, 0.01% NAN₃, pH 7.2, using a protein concentration of 5 micrograms per ml. All volumes in ELISA plates are 50 microliters, except for storage, where 200 microliters is added. After coating, plates are incubated with 10 mM tris base, 150 mM NaCl, pH 7.4 CIBS) containing 2% bovine serum albumin (BSA) and stored at 4oC. Plates prepared this way are stable for several weeks.

A peptide derived from the protein tau was utilized, as shown below:
Biotin-KKVAVR(phospho)TPPKSPSS (SEQ D NO. 1)
   The peptide was diluted to a concentration of 0.5 micromolar in TBS containing 2% BSA, and 50 microliters per well were added followed by incubation at room temperature for one hour. Unbound peptide is washed off with TBS containing 0.5% Tween 20. Compounds to be tested for binding are mixed with TBS at concentrations ranging from 100 micromolar to 0.01 nanomolar, and 50 microliters is added to each well. After one hour at room temperature, unbound compounds are removed by aspiration. A solution of an antibody specifically reactive with the phosphoepitope is added to each well of the plate. Antibodies useful in this regard include the monoclonal antibodies AT180, TG3, CP9, CP10, CP16, CP17, GF1, GF3, GF5, GF25, and GF31. Polyclonal antibodies produced to phosphopeptides containing sequences substantially similar to that above may also be useful. After 30 minutes at room temperature, unbound antibody is removed by washing with TBS containing 0.05% Tween 20. Bound antibody is detected by incubation with a solution of goat anti-mouse Ig or goat anti-rabbit Ig coupled to horse radish peroxidase (HRP) (2 micrograms per ml in TBS containing 1% BSA) for one hour, and excess antibody is removed by washing with TBS containing 0.05% Tween 20. HRP activity is determined by addition of a substrate capable of generating a colored or fluorescent product (i.e., ABTS Substrate solution (BioRad Laboratories, Hercules, Calif.)). Compounds bound to the phosphorylated epitope of the peptide prevent access of the antibody to this site, and decrease the color formation.

The specificity of binding of the compounds to phosphoditeonine 231 of tau is established using the same methods except that the tau 231 phosphopeptide is replaced with irrelevant phosphopeptides derived from the sequence of tau or other proteins, and compound binding to these sequences is determined with the appropriate antibodies. Examples of useful phosphopeptide /monoclonal antibody combinations are shown below:

ATRIPAK(phospho)TPPAPKTP (tau175P; SEQ ID NO. 4),
bound by CP18
SGYSSPG(phospho)SPGTPGSR (tau202P; SEQ ID NO. 5),
bound by CP13
GSRSRTP(phospho)SLPTPPTR (tau214P; SEQ ID NO. 6),
bound by CP3
DTSPRHL(phospho)SNVSSTGS (tau409P; SEQ ID NO. 7),
bound by PG5

These phosphopeptides may be used in place of the tau231P peptide using the same methodology as described above, using the appropriate antibody, and may optionally be biotinylated. Compounds specifically bound to tau231P will not bind to SEQ ID Nos. 5-8, and thus will not reduce the binding of the appropriate antibody.

It is also possible to practice the method described above using a phosphopeptide corresponding to the sequence of APP. The sequence of one such peptide is shown below:
Biotin-VEVDAAV(phospho)TPEERHLS (SEQ ID NO. 2)
   In place of the TG3 antibody, a mAb specific for the phosphothreonine 668 of APP is used. The same antibody detection reagents and methods are used as described above. Examples of antibodies useful in this regard include but are not limited to GF1, GF3, GF5, GF7, GF12, GF25, and GF31. Polyclonal antibodies reactive against substantially similar phosphopeptides may also be useful.

Another method relates to phosphopeptides derived from cdc25. The same method as described above is utilized except that the phosphopeptide is derived from cdc25, such as

| | |
|---|---|
| Biotin-VCPDVPR(phospho)TPVGKFLG | (SEQ ID NO. 3) |

In place of the TG3 antibody, a mAb specific for the phosphothreonine 48 of cdc25 is used, along with the same antibody detection reagents and methods. Examples of antibodies useful in this regard include but are not limited to GF1 and GF25. Polyclonal antibodies reactive with substantially similar phosphoepitopes may also be useful.
B. Experimental Results

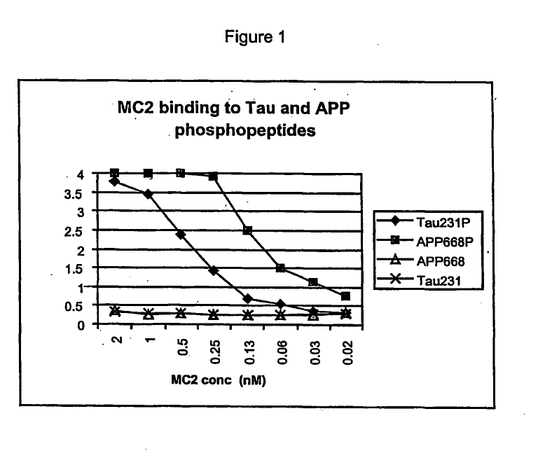

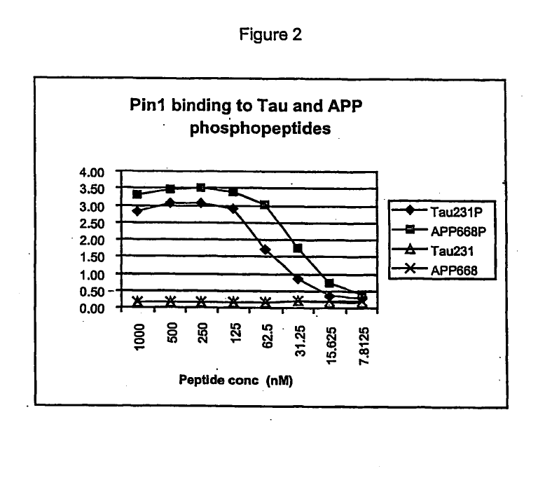

That structural similarities exist between the region of tau near threonine 231 (thr231) and of APP near threonine 668 (thr668) was first demonstrated by the binding of a monoclonal antibody, MC2, to synthetic phosphopeptides comprising both sites (FIG. 1). Similarly, the protein Pin1 binds to phosphopeptides derived from both proteins (FIG. 2). This data provides very strong evidence for structural similarity between these two regions from proteins which otherwise have little or no sequence homology. These regions of the proteins must adopt similar conformations, and there is published evidence that the region of the APP protein around threonine 668 adopts a reverse beta turn structure (Shen, et al. The essential mitotic peptidyl-prolyl isomerase Pin1 binds and regulates mitosis-specific phosphoproteins. Genes Dev. 12, 706-720. 1998), and evidence has been obtained that the same structural feature is found in the region of tau around threonine 231.

To further study the thr231 and thr668 regions of the tau and APP proteins, respectively, monoclonal antibodies were raised to both the phosphothreonine 231 site of tau, and the phosphothreonine 668 site of APP using synthetic phosphopeptides linked to KLH as is known to one skilled in the art. An antibody to the thr668P site has been described (Suzuki, et al. Cell cycle-dependent regulation of the phosphorylation and metabolism of the Alzheimer amyloid precursor protein. EMBO J. 13:1114-1122, 1994).

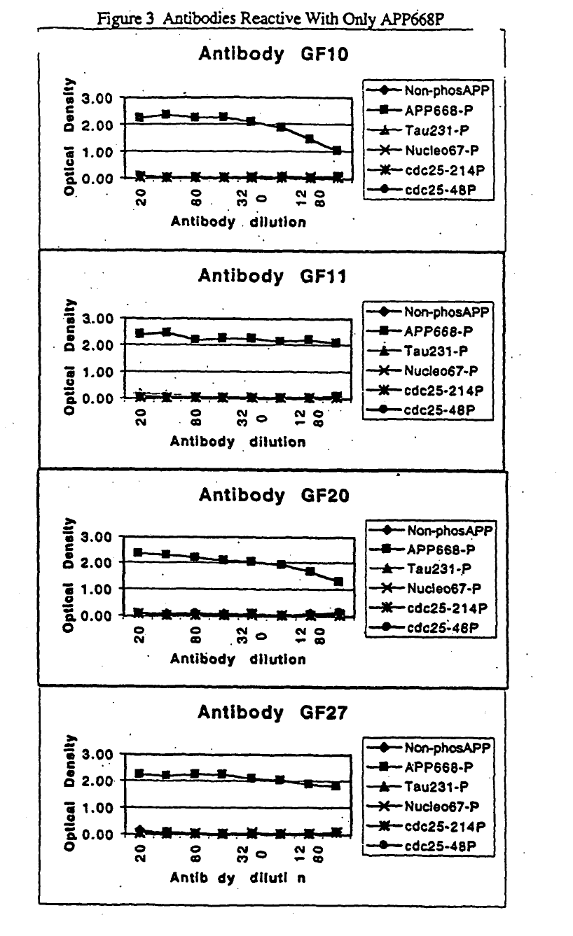

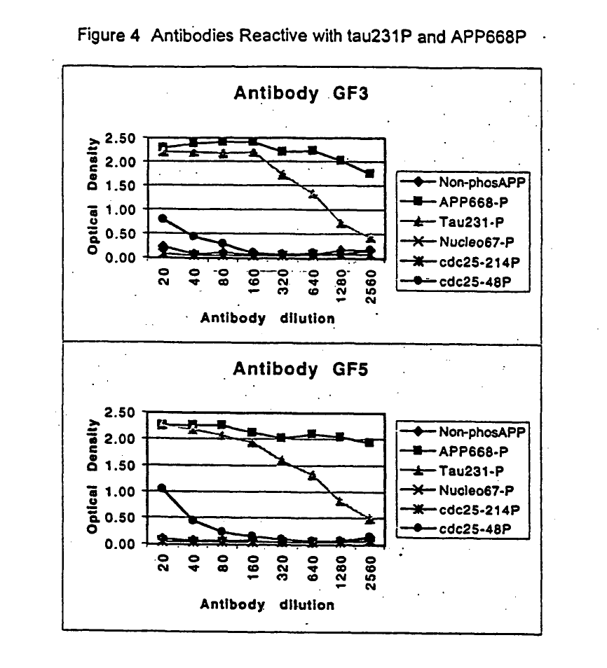

The antibodies GF10, GF11, GF20 and GP27 specifically recognize the APP668 phosphothreonine phosphopeptide without recognition of the tau231 phosphopeptide (FIG. 3). Additional “MC2-like” antibodies, namely GF3 and GF5, were found to recognize both sequences (FIG. 4). Another series of antibodies is available that specifically recognizes tau231P encompassed by AT180, TG3, CP9, CP10, CP16, CP17, GF1, and GP25.

Certain of the antibodies described herein (i.e., GP3, GF5, and GF31) recognized both the thr231P and thr668P sites, but not several other phosphopeptides tested. These results suggest that despite the lack of amino acid sequence homology between these sites, these phosphoepitopes most likely share similarities in conformation. NMR studies of peptide conformations from these regions of tau and APP are suggestive of an unusual reverse beta turn in the structures of both peptides (Kroenke, et al. Solution conformations of a peptide containing the cytoplasmic domain sequence of the beta amyloid precursor protein. Biochemistry 36, 8145-8152, 1997; Weaver, et al. Conformational Requirements For The Monoclonal Antibody TG3 Reactivity And Specificity For Alzheimer's Disease Elucidated Through NMR Spectroscopy. Society for Neuroscience, Abstract #448.10, 1999).

Monoclonal antibody GF7 to the APP thr668 phosphoepitope show specific staining of brain tissues from cases of AD. Labeling is intraneuronal, and was found in the same regions that are known to show staining with the tau 231 phosphoepitope-specific antibody TG3. Double labeling immunocytochemistry using tissues from early AD cases show that both phosphoepitopes accumulate in the same neurons of the hippocampus (data not shown). This data strongly suggests that both tau and APP are phosphorylated by cdc2 (or a similar kinase) early in the course of Alzheimer's disease.

### Example 2

### Assay to Identify Compounds that Bind Tau231P and/or APP668P

Due to the apparent similarity between the tau and APP phosphoepitopes, studies were conducted to determine whether Pin1 will bind to both proteins after phosphorylation by cdc2. Phosphopeptides derived from both the tau sequence near threonine 231 and around APP threonine 668 both bind Pin1 with high affinity. This fact has led to the development of assays for identifying binding agents that interfere with the interaction of Pin 1 with tau and APP.

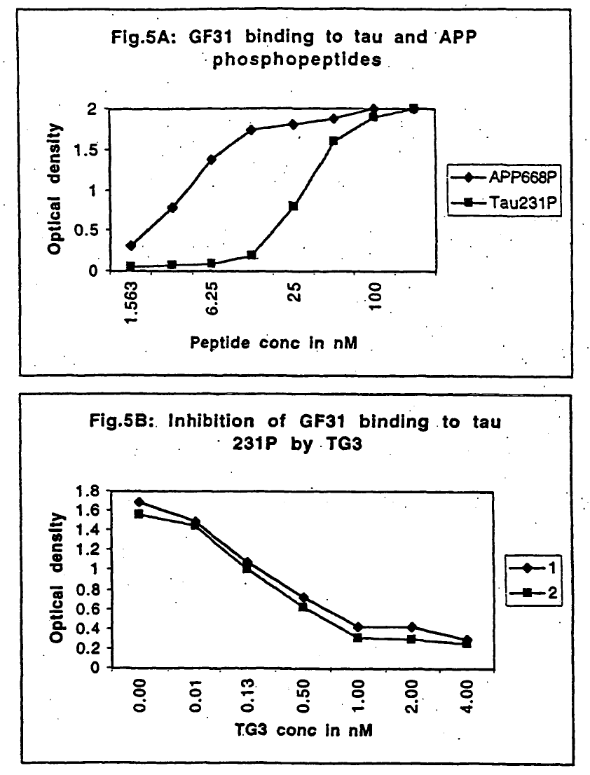

Assays have been established in which the tau and APP phosphopeptides are biotinylated and immobilized on Neutravidin-coated 96 well ELISA plates. The GF31 antibody recognizes both the tau231P and APP668P phosphopeptides, with a lower affinity for tau231P (FIG. 5A). This lower affinity for the tau phosphopeptide has been exploited by developing an extremely sensitive assay in which low concentrations of this biotinylated peptide (30 nM) are incubated with neutravidin-coated 96 well plates. Concentrations of GF31 in the 5 nM range give a robust signal on such plates, and the binding is very sensitive to the presence of an antibody, TG3, which has a high affinity for the tau peptide (FIG. 5B). Duplicate assays are shown in FIG. 5B, illustrating the reproducibility of the assay. The affinity of TG3 for the tau phosphopeptide has been estimated from other studies to be about 25 nM, and inhibition is readily detected with concentrations of the antibody two orders of magnitude below this concentration.

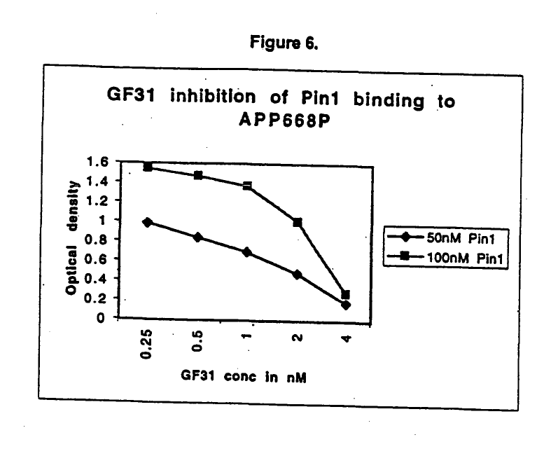

A second stage assay is also provided and is useful for further screening binding agents discovered in the GF31/tau231P assay. This assay is useful for confirming that binding agents which inhibit the binding of GF31 to the tau231P peptide also block Pin1 binding to both the tau and APP phosphopeptides. This assay examines Pin1 binding to either the tau231P or the APP668P peptides (FIG. 6). The appropriate concentrations of tau231P, APP668P and Pin1 for use in this assay have been determined. The data shows Pin1 binding at two different concentrations with the APP668P peptide. As the figure shows, it is possible to demonstrate that GF31 is an inhibitor compound. Thus, the assay is robust and sensitive to the presence of low concentrations of compounds that bind to the APP668P peptide.

It is clear that techniques developed to discover compounds which bind to specific Pin1 binding sites on phosphorylated tau and/or APP could equally well be applied to discover compounds that bind to and block Pin1 binding to phosphorylated sites on cdc25. For an anticancer screen, the cdc25-48P peptide is used: biotin-VCPDVPR(phospho)TPVGKFLG (SEQ ID NO. 3), with an appropriate antibody (i.e., GF1 or GF25). Thus, monoclonal antibodies have been produced which recognize Pin1 binding sites on all three proteins. Binding agents that block binding of these antibodies (i.e., binding surrogates) to the appropriate cdc25 sites would provide a novel approach to the development of compounds that interfere with mitosis.

While a preferred form of the invention has been shown in the drawings and described, since variations in the preferred form will be apparent to those skilled in the art, the invention should not be construed as limited to the specific form shown and described, but instead is as set forth in the claims.

1. A method for identifying a desired binding agent that interferes with the interaction between a protein, protein fragment, polypeptide or peptide and a binding surrogate, comprising:
a) combining the protein, protein fragment, polypeptide or peptide, the binding surrogate, and a test binding agent to form a reaction mixture;
b) detecting binding between the protein, protein fragment, polypeptide or peptide and the binding surrogate;
wherein the decrease of interaction between the protein, protein fragment, polypeptide or peptide and the binding surrogate indicates that the test binding agent interferes with the interaction.

2. The method of claim 1 further comprising step c) wherein the level of interaction between the protein, protein fragment, polypeptide or peptide and the binding surrogate in the absence of the test binding agent is determined as a control and compared to the level of interaction detected in step b); whereby a desired binding agent results in a decreased level of interaction in step b) as compared to the level of interaction detected in step c).

3. The method of claim 1 wherein said protein, protein fragment, polypeptide or peptide is selected from the group consisting of a protein, protein fragment, polypeptide and peptide comprising amino acid sequence corresponding to tau231P, APP668P, or cdc25-48P.

4. The method of claim 1 wherein said protein, protein fragment, polypeptide or peptide is a peptide of about 15 or less amino acids, said peptide comprising at least one region of identity to tau231P, APP668P, or cdc25-48P.

5. The method of claim 1 wherein said protein, protein fragment, polypeptide or peptide is a peptide of about 15 or less amino acids, said peptide comprising at least one phosphorylated threonine residue.

6. The method of claim 1 wherein said binding surrogate is selected from the group consisting of a peptide, polypeptide, protein, monoclonal antibody, polyclonal antibody, fragment thereof, and derivative thereof.

7. The method of claim 1 wherein said binding surrogate is selected from the group consisting of the monoclonal antibody AT180, TG3, CP9, CP10, CP16, CP17, GF1, GF7, GF25, MC2, GF10, GF11, GF20, GF27, GP3GF5, GF31, a fragment thereof, and a derivative thereof.

8. The method of claim 1 wherein said polypeptide is selected from the group consisting of Pin 1, a fragment thereof, a derivative thereof, and a peptide corresponding to Pin 1.

9. The method of claim 2 wherein said protein, protein fragment, polypeptide or peptide is selected from the group consisting of a protein, protein fragment, polypeptide or peptide comprising amino acid sequence corresponding to tau231P, APP668P, or cdc25-48P.

10. The method of claim 2 wherein said protein, protein fragment, polypeptide or peptide is a peptide of about 15 or less amino acids, said peptide comprising at least one region of identity to tau231P, APP668P, or cdc25-48P.

11. The method of claim 2 wherein said protein, protein fragment, polypeptide or peptide is a peptide of about 15 or less amino acids, said peptide comprising at least one phosphorylated threonine residue.

12. The method of claim 2 wherein said binding surrogate is selected from the group consisting of a peptide, polypeptide, protein, monoclonal antibody, polyclonal antibody, fragment thereof, and derivative thereof.

13. The method of claim 2 wherein said binding surrogate is selected from the group consisting of the monoclonal antibody AT180, TG3, CP9, CP10, CP16, CP17, GF1, GF7, GP25, MC2, GF10, GF11, GF20, GF27, GF3, GF5, GF31, a fragment thereof, and a derivative thereof.

14. The method of claim 2 wherein said polypeptide is selected from the group consisting of Pin 1, a fragment thereof, a derivative thereof, and a peptide corresponding to Pin 1.

15. A binding agent identified by the method of claim 1.

16. A binding agent of claim 15 wherein said binding agent is a small organic molecule.

17. A binding agent of claim 16 wherein said binding agent is a small organic molecule useful for preventing or treating Alzheimer's Disease.

18. A binding agent of claim 16 wherein said binding agent is a small organic molecule useful for preventing or treating cancer.

19. A binding agent identified by the method of claim 2.

20. A binding agent of claim 19 wherein said binding agent is a small organic molecule.

21. A binding agent of claim 20 wherein said binding agent is a small organic molecule useful for preventing or treating Alzheimer's Disease.

22. A binding agent of claim 20 wherein said binding agent is a small organic molecule useful for preventing or treating cancer.

23. The method of claim 1 wherein said protein, protein fragment, polypeptide or peptide is labeled with biotin.

24. The method of claim 2 wherein said protein, protein fragment, polypeptide or peptide is labeled with biotin.

25. The method of claim 1 wherein said protein, protein fragment, polypeptide or peptide is labeled with biotin and said reaction mixture is within a reaction vessel; said reaction vessel being pre-coated with avidin.

26. The method of claim 2 wherein said protein, protein fragment, polypeptide or peptide is labeled with biotin and said reaction mixture is within a reaction vessel; said reaction vessel being precoated with avidin.

27. The method of claim 1 wherein the interaction in step a) is determined by measuring the amount of binding surrogate bound to the protein, protein fragment, polypeptide or peptide.

28. The method of claim 2 wherein the interaction in step a) is determined by measuring the amount of binding surrogate bound to the protein, protein fragment, polypeptide or peptide.

29. A method for identifying a binding agent that interferes with the interaction between at least one protein, protein fragment, polypeptide or peptide and a binding surrogate comprising:
a) combining a first protein, protein fragment, polypeptide or peptide with a binding surrogate to form a reaction mixture under conditions in which the peptide and the binding surrogate interact with one another;
b) introducing a test binding agent into the reaction mixture;
c) detecting the level of interaction between the protein, protein fragment, polypeptide or peptide, and the binding surrogate;
wherein a decrease in the level of interaction between the protein, protein fragment, polypeptide or peptide and the binding surrogate indicates that the test binding agent interferes with the interaction.

30. The method of claim 29 further comprising step d) wherein the level of interaction between the protein, protein fragment, polypeptide. or peptide and the binding surrogate in the absence of the test binding agent is determined as a control and compared to the level of interaction detected in step c); whereby a desired binding agent results in a decreased level of interaction in step c) as compared to the level of interaction detected in step d).

31. The method of claim 29 wherein said protein, protein fragment, polypeptide or peptide is selected from the group consisting of a protein, protein fragment, polypeptide or peptide comprising amino acid sequence corresponding to tau231P, APP668P, and cdc25-48P.

32. The method of claim 29 wherein said protein, protein fragment, polypeptide or peptide is a peptide of about 15 or less amino acids, said peptide comprising at least one region of identity to tau231P, APP668P, or cdc25-48P.

33. The method of claim 29 wherein said protein, protein fragment, polypeptide or peptide is a peptide of about 15 or less amino acids, said peptide comprising at least one phosphorylated threonine residue.

34. The method of claim 29 wherein said binding surrogate is selected from the group consisting of a peptide, polypeptide, protein, monoclonal antibody, polyclonal antibody, fragment thereof, and derivative thereof.

35. The method of claim 29 wherein said binding surrogate is selected from the group consisting of the monoclonal antibody AT180, TG3, CP9, CP10, CP16, CP17, GP1, GP7, GP25, MC2, GP10, GF11, GF20, GF27, GF3, GF5, GF31, a fragment thereof, and a derivative thereof.

36. The method of claim 29 wherein said polypeptide is selected from the group consisting of Pin 1, a fragment thereof, a derivative thereof, and a peptide corresponding to Pin 1.

37. The method of claim 30 wherein said protein, protein fragment, polypeptide or peptide is selected from the group consisting of a protein, protein fragment, polypeptide or peptide comprising amino acid sequence corresponding to tau231P, APP668P, or cdc25-48P.

38. The method of claim 30 wherein said protein, protein fragment, polypeptide or peptide is a peptide of about 15 or less amino acids, said peptide comprising at least one region of identity to tau231P, APP668P, or cdc25-48P.

39. The method of claim 30 wherein said protein, protein fragment, polypeptide or peptide is a peptide of about 15 or less amino acids, said peptide comprising at least one phosphorylated threonine residue.

40. The method of claim 30 wherein said binding surrogate is selected from the group consisting of a peptide, polypeptide, protein, monoclonal antibody, polyclonal antibody, fragment thereof, and derivative thereof.

41. The method of claim 30 wherein said binding surrogate is selected from the group consisting of the monoclonal antibody AT180, TG3, CP9, CP10, CP16, CP17, GF1, GF7, GF25, MC2, GF10, GF11, GF20, GP27, GF3, GF5, GF31, a fragment thereof, and a derivative thereof.

42. The method of claim 30 wherein said polypeptide is selected from the group consisting of Pin 1, a fragment thereof, a derivative thereof, and a peptide corresponding to Pin 1.

43. A binding agent identified by the method of claim 29.

44. A binding agent of claim 43 wherein said binding agent is a small organic molecule.

45. A binding agent of claim 44 wherein said binding agent is a small organic molecule useful for preventing or treating Alzheimer's Disease.

46. A binding agent of claim 44 wherein said binding agent is a small organic molecule useful for preventing or treating cancer.

47. A binding agent identified by the method of claim 30.

48. A binding agent of claim 47 wherein said binding agent is a small organic molecule.

49. A binding agent of claim 48 wherein said binding agent is a small organic molecule useful for preventing or treating Alzheimer's Disease.

50. A binding agent of claim 48 wherein said binding agent is a small organic molecule useful for preventing or treating cancer.

51. The method of claim 29 wherein said protein, protein fragment, polypeptide or peptide is labeled with biotin.

52. The method of claim 30 wherein said protein, protein fragment, polypeptide or peptide is labeled with biotin.

53. The method of claim 29 wherein said protein, protein fragment, polypeptide or peptide is labeled with biotin and said reaction mixture is within a reaction vessel; said reaction vessel being pre-coated with avidin.

54. The method of claim 30 wherein said protein, protein fragment, polypeptide or peptide is labeled with biotin and said reaction mixture is within a reaction vessel; said reaction vessel being pre-coated with avidin.

55. The method of claim 29 wherein the interaction in step a) is determined by measuring the amount of binding surrogate bound to the protein, protein fragment, polypeptide or peptide.

56. The method of claim 30 wherein the interaction in step a) is determined by measuring the amount of binding surrogate bound to the protein, protein fragment, polypeptide or peptide.

57. A method for identifying a binding agent that interferes with the interaction between at least one protein, protein fragment, polypeptide or peptide and a binding surrogate comprising:
a) combining a protein, protein fragment, polypeptide or peptide with a test binding agent to form a reaction mixture under conditions in which the peptide and the binding surrogate interact with one another,
b) introducing a binding surrogate into the reaction mixture;
c) detecting the level of interaction between the protein, protein fragment, polypeptide or peptide and the binding surrogate;
wherein a decrease in the level of interaction between the protein, protein fragment, polypeptide or peptide and the binding surrogate indicates that the test binding agent interferes with the interaction.

58. The method of claim 57 further comprising step d) wherein the level of interaction between the protein, protein fragment, polypeptide or peptide and the binding surrogate in the absence of the test binding agent is determined as a control and compared to the level of interaction detected in step c); whereby a desired binding agent results in a decreased level of interaction in step c) as compared to the level of interaction detected in step d).

59. The method of claim 57 wherein said protein, protein fragment, polypeptide or peptide is selected from the group consisting of a protein, protein fragment, polypeptide or peptide comprising amino acid sequence corresponding to tau231P, APP668P, or cdc25-48P.

60. The method of claim 57 wherein said protein, protein fragment, polypeptide or peptide is a peptide of about 15 or less amino acids, said peptide comprising at least one region of identity to tau231P, APP668P, or cdc25-48P.

61. The method of claim 57 wherein said protein, protein fragment, polypeptide or peptide is a peptide of about 15 or less amino acids, said peptide comprising at least one phosphorylated threonine residue.

62. The method of claim 57 wherein said binding surrogate is selected from the group consisting of a peptide, polypeptide, protein, monoclonal antibody, polyclonal antibody, fragment thereof, and derivative thereof.

63. The method of claim 57 wherein said binding surrogate is selected from the group consisting of the monoclonal antibody AT180, TG3, CP9, CP10, CP16, CP17, GF1, GF7, GF25, MC2, GF10, GF11, GF20, GF27, GF3, GF5, GF31, a fragment thereof, and a derivative thereof.

64. The method of claim 57 wherein said polypeptide is selected from the group consisting of Pin 1, a fragment thereof, a derivative thereof, and a peptide corresponding to Pin 1.

65. The method of claim 57 wherein said protein, protein fragment, polypeptide or peptide is selected from the group consisting of a protein, protein fragment, polypeptide or peptide comprising amino acid sequence corresponding to tau231P, APP668P, or cdc25-48P.

66. The method of claim 57 wherein said protein, protein fragment, polypeptide or peptide is a peptide of about 15 or less amino acids, said peptide comprising at least one region of identity to tau231P, APP668P, or cdc25-48P.

67. The method of claim 57 wherein said protein, protein fragment, polypeptide or peptide is a peptide of about 15 or less amino acids, said peptide comprising at least one phosphorylated threonine residue.

68. The method of claim 58 wherein said binding surrogate is selected from the group consisting of a peptide, polypeptide, protein, monoclonal antibody, polyclonal antibody, fragment thereof, and derivative thereof.

69. The method of claim 58 wherein said binding surrogate is selected from the group consisting of the monoclonal antibody AT180, TG3, CP9, CP10, CP16, CP17, GP1, GF7, GF25, MC2, GF10, GF11, GF20, GF27, GF3, GF5, GF31, a fragment thereof, and a derivative thereof.

70. The method of claim 58 wherein said polypeptide is selected from the group consisting of Pin 1, a fragment thereof, a derivative thereof, and a peptide corresponding to Pin 1.

71. A binding agent identified by the method of claim 57.

72. A binding agent of claim 71 wherein said binding agent is a small organic molecule.

73. A binding agent of claim 72 wherein said binding agent is a small organic molecule useful for preventing or treating Alzheimer's Disease.

74. A binding agent of claim 72 wherein said binding agent is a small organic molecule useful for preventing or treating cancer.

75. A binding agent identified by the method of claim 58.

76. A binding agent of claim 75 wherein said binding agent is a small organic molecule.

77. A binding agent of claim 76 wherein said binding agent is a small organic molecule useful for preventing or treating Alzheimer's Disease.

78. A binding agent of claim 76 wherein said binding agent is a small organic molecule useful for preventing or treating cancer.

79. The method of claim 57 wherein said protein, protein fragment, polypeptide or peptide is labeled with biotin.

80. The method of claims 58 wherein said protein, protein fragment, polypeptide or peptide is labeled with biotin.

81. The method of claim 57 wherein said protein, protein fragment, polypeptide or peptide is labeled with biotin and said reaction mixture is within a reaction vessel; said reaction vessel being pre-coated with avidin.

82. The method of claims 58 wherein said protein, protein fragment, polypeptide or peptide is labeled with biotin and said reaction mixture is within a reaction vessel; said reaction vessel being pre-coated with avidin.

83. The method of claim 57 wherein the interaction in step a) is determined by measuring the amount of binding surrogate bound to the protein, protein fragment, polypeptide or peptide.

84. The method of claim 58 wherein the interaction in step a) is determined by measuring the amount of binding surrogate bound to the protein, protein fragment, polypeptide or peptide.

85. A method for identifying a binding agent that interferes with the interaction between at least one protein, protein fragment, polypeptide or peptide and a binding surrogate comprising:
a) combining a binding surrogate with a test binding agent to form a reaction mixture under conditions in which the peptide and the binding surrogate interact with one another;
b) introducing a one protein, protein fragment, polypeptide or peptide into the reaction mixture;
c) detecting the level of interaction between the protein, protein fragment, polypeptide or peptide and the binding surrogate;
wherein a decrease in the level of interaction between the protein, protein fragment, polypeptide or peptide and the binding surrogate indicates that the test binding agent interferes with the interaction.

86. The method of claim 85 further comprising step d) wherein the level of interaction between the protein, protein fragment, polypeptide or peptide and the binding surrogate in the absence of the test binding agent is determined as a control and compared to the level of interaction detected in step c); whereby a desired binding agent results in a decreased level of interaction in step c) as compared to the level of interaction detected in step d).

87. The method of claim 85 wherein said protein, protein fragment, polypeptide or peptide is selected from the group consisting of a protein, protein fragment, polypeptide or peptide comprising amino acid sequence corresponding to tau231P, APP668P, or cdc25-48P.

88. The method of claim 85 wherein said protein, protein fragment, polypeptide or peptide is a peptide of about 15 or less amino acids, said peptide comprising at least one region of identity to tau231P, APP668P, or cdc25-48P.

89. The method of claim 85 wherein said protein, protein fragment, polypeptide or peptide is a peptide of about 15 or less amino acids, said peptide comprising at least one phosphorylated threonine residue.

90. The method of claim 85 wherein said binding surrogate is selected from the group consisting of a peptide, polypeptide, protein, monoclonal antibody, polyclonal antibody, fragment thereof, and derivative thereof.

91. The method of claim 85 wherein said binding surrogate is selected from the group consisting of the monoclonal antibody AT180, TG3, CP9, CP10, CP16, CP17, GP1, GF7, GF25, MC2, GF10, GF11, GP20, GF27, GF3, GF5, GP31, a fragment thereof, and a derivative thereof.

92. The method of claim 85 wherein said polypeptide is selected from the group consisting of Pin 1, a fragment thereof, a derivative thereof, and a peptide corresponding to Pin 1.

93. The method of claim 85 wherein said protein, protein fragment, polypeptide or peptide is selected from the group consisting of a protein, protein fragment, polypeptide or peptide comprising amino acid sequence corresponding to tau231P, APP668P, or cdc25-48P.

94. The method of claim 85 wherein said protein, protein fragment, polypeptide or peptide is a peptide of about 15 or less amino acids, said peptide comprising at least one region of identity to tau231P, APP668P, or cdc25-48P.

95. The method of claim 85 wherein said protein, protein fragment, polypeptide or peptide is a peptide of about 15 or less amino acids, said peptide comprising at least one phosphorylated threonine residue.

96. The method of claim 86 wherein said binding surrogate is selected from the group consisting of a peptide, polypeptide, protein, monoclonal antibody, polyclonal antibody, fragment thereof, and derivative thereof.

97. The method of claim 86 wherein said binding surrogate is selected from the group consisting of the monoclonal antibody AT180, TG3, CP9, CP10, CP16, CP17, GF1, GF7, GF25, MC2, GF10, GF11, GF20, GF27, GF3, GF5, GP31, a fragment thereof, and a derivative thereof.

98. The method of claim 86 wherein said polypeptide is selected from the group consisting of Pin 1, a fragment thereof, a derivative thereof, and a peptide corresponding to Pin 1.

99. A binding agent identified by the method of claim 85.

100. A binding agent of claim 99 wherein said binding agent is a small organic molecule.

101. A binding agent of claim 100 wherein said binding agent is a small organic molecule useful for preventing or treating Alzheimer's Disease.

102. A binding agent of claim 100 wherein said binding agent is a small organic molecule useful for preventing or treating cancer.

103. A binding agent identified by the method of claim 86.

104. A binding agent of claim 103 wherein said binding agent is a small organic molecule.

105. A binding agent of claim 104 wherein said binding agent is a small organic molecule useful for preventing or treating Alzheimer's Disease.

106. A binding agent of claim 104 wherein said binding agent is a small organic molecule useful for preventing or treating cancer.

107. The method of claims 85 wherein said protein, protein fragment, polypeptide or peptide is labeled with biotin.

108. The method of claims 86 wherein said protein, protein fragment, polypeptide or peptide is labeled with biotin.

109. The method of claims 85 wherein said protein, protein fragment, polypeptide or peptide is labeled with biotin and said reaction mixture is within a reaction vessel; said reaction vessel being pre-coated with avidin.

110. The method of claims 86 wherein said protein, protein fragment, polypeptide or peptide is labeled with biotin and said reaction mixture is within a reaction vessel; said reaction vessel being pre-coated with avidin.

111. The method of claim 85 wherein the interaction in step a) is determined by measuring the amount of binding surrogate bound to the protein, protein fragment, polypeptide or peptide.

112. The method of claim 85 wherein the interaction in step a) is determined by measuring the amount of binding surrogate bound to the protein, protein fragment, polypeptide or peptide.
